SEQUENCE LISTING (12 sequences)

SEQ ID NO: 1 - DNA, 705 bp, Homo sapiens
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtc

SEQ ID NO: 2 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 3 - DNA, 1131 bp, Pyrocystis lunula
atggtgtgtgagaagggattcgaggtgggtgacaacgtcaagggcggccccctgaactccaagcagctcgaaaaatacggcgacaacttcaaggacggcatgcaccaacctaccttccacgacgaaggtctccacaaacctatggaggctggaggcaaaaccttcgagagcggattccactacctcctggagtgccatgagctggggggcaagaacgccaccggtggctacgggggacctctctgcgaggacccttacggagctgaggtgtccaagctggtggatcaggtcctgaaggactccgacagcgaccggacactctgctacaacaacttccacgacccttgccctgagctgaccaaggggcaagtggctatgtgcaagggattcgactacggcgacaagaccctgaagctgccttgtgggccactgccctggcccgccggctgtcctgaacctggatacgtgcctaagaccaacccccttcacggtcgctggatcaccgtgtccggcggacaagctgctttcattaaagaggccatcaagagcggaatgctgggtgctgccgaggccaacaagatcgctgccgacaccgaccacgagcagaccggcagcatgtttctccgcatcaatcagttcggcgatcagtgcaccgtggacgcctccgtggctaagtacgctcgcgccaagcgcacctggaggtccggtcactacttctacgagcccctggtctccggcgggaacctcctgggcgtgtgggtgctgcctgaggagtaccgcaagatcgggtttttctgggaaatggagtccggtcggtgctttcgcatcgagagaagggccttccctgtcggcccttacacctttctgcgccaggctaccgaggtgaacggcacaattagcttcgtgctgtacgtgaaggtgtccaacgaccctgagtccaagcctatccccgtgcagagccgcgactacactgccctggctggttgcgacaacgtctgcaccaacctgggaaagccttatccttgcaccgctaaagacctggactaccctaacaagcgcgacacttggctcgatcagaacgagaaggagatgatccatcagagaggccttgtggctaccagcttcaaggcc

SEQ ID NO: 4 - protein, 377 aa, Pyrocystis lunula
MVCEKGFEVGDNVKGGPLNSKQLEKYGDNFKDGMHQPTFHDEGLHKPMEAGGKTFESGFHYLLECHELGGKNATGGYGGPLCEDPYGAEVSKLVDQVLKDSDSDRTLCYNNFHDPCPELTKGQVAMCKGFDYGDKTLKLPCGPLPWPAGCPEPGYVPKTNPLHGRWITVSGGQAAFIKEAIKSGMLGAAEANKIAADTDHEQTGSMFLRINQFGDQCTVDASVAKYARAKRTWRSGHYFYEPLVSGGNLLGVWVLPEEYRKIGFFWEMESGRCFRIERRAFPVGPYTFLRQATEVNGTISFVLYVKVSNDPESKPIPVQSRDYTALAGCDNVCTNLGKPYPCTAKDLDYPNKRDTWLDQNEKEMIHQRGLVATSFKA

SEQ ID NO: 5 - DNA, 1956 bp, Artificial
  DNA encoding a monitor protein in pCMV-DL-D3-CD8
atgaccatgtctgcacttctgatcctagctcttgttggagctgcagttgctgactacaaagaccatgacggtgattataaagatcatgacatcgattacaaggatgacgatgacaagcttgtgtgtgagaagggattcgaggtgggtgacaacgtcaagggcggccccctgaactccaagcagctcgaaaaatacggcgacaacttcaaggacggcatgcaccaacctaccttccacgacgaaggtctccacaaacctatggaggctggaggcaaaaccttcgagagcggattccactacctcctggagtgccatgagctggggggcaagaacgccaccggtggctacgggggacctctctgcgaggacccttacggagctgaggtgtccaagctggtggatcaggtcctgaaggactccgacagcgaccggacactctgctacaacaacttccacgacccttgccctgagctgaccaaggggcaagtggctatgtgcaagggattcgactacggcgacaagaccctgaagctgccttgtgggccactgccctggcccgccggctgtcctgaacctggatacgtgcctaagaccaacccccttcacggtcgctggatcaccgtgtccggcggacaagctgctttcattaaagaggccatcaagagcggaatgctgggtgctgccgaggccaacaagatcgctgccgacaccgaccacgagcagaccggcagcatgtttctccgcatcaatcagttcggcgatcagtgcaccgtggacgcctccgtggctaagtacgctcgcgccaagcgcacctggaggtccggtcactacttctacgagcccctggtctccggcgggaacctcctgggcgtgtgggtgctgcctgaggagtaccgcaagatcgggtttttctgggaaatggagtccggtcggtgctttcgcatcgagagaagggccttccctgtcggcccttacacctttctgcgccaggctaccgaggtgaacggcacaattagcttcgtgctgtacgtgaaggtgtccaacgaccctgagtccaagcctatccccgtgcagagccgcgactacactgccctggctggttgcgacaacgtctgcaccaacctgggaaagccttatccttgcaccgctaaagacctggactaccctaacaagcgcgacacttggctcgatcagaacgagaaggagatgatccatcagagaggccttgtggctaccagcttcaaggccaagcttgccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtc

SEQ ID NO: 6 - protein, 652 aa, Artificial [sequence omitted]

SEQ ID NO: 7 - DNA, 12 bp, Artificial
  synthetic DNA
aaggacgagctg

SEQ ID NO: 8 - protein, 4 aa, Artificial
  Amino acid sequence encoded by DNA of SEQ ID NO 7
KDEL

SEQ ID NO: 9 - protein, 13 aa, Artificial
  Spacer
PPPPPPPPPPPPP

SEQ ID NO: 10 - protein, 5 aa, Artificial
  Spacer
GGGGG

SEQ ID NO: 11 - protein, 10 aa, Artificial
  Spacer
NNNNNNNNNN

SEQ ID NO: 12 - protein, 17 aa, Artificial
  Spacer
SSSSSSSSSSSSSSSSS

(19) United States
(12) Patent Application Publication
(10) Pub. No.: US 2008/0182274 A1
(43) Pub. Date: Jul. 31, 2008

(54) MONITOR PROTEINS USABLE FOR ANALYZING EXPRESSION OF MEMBRANE PROTEINS

(75) Inventors: Yoshiro Hanyu, lbaraki (JP); Mieko Kato, Ibaraki (JP); Akihiro Kurita, Tokyo (JP); Yutaka Yamagishi, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignees: National Institute of Advanced Industrial Science and Technology; Toyo B-Net Co., Ltd.

(21) Appl. No.: 12/016,971

(22) Filed: Jan. 18, 2008

(30) Foreign Application Priority Data
Jan. 22, 2007 (JP) 2007-011163

(51) Int. Cl. G01N 33/53; C12N 15/11; C12N 15/00; C12N 5/06

(52) U.S. Cl. 435/7.2; 536/23.4; 435/320.1; 435/325

(57) ABSTRACT

A monitor protein for measuring the expression of a membrane protein on a cell membrane is provided. The monitor protein includes a fusion protein in which the membrane protein and a luminescence protein are linked. Also provided are a DNA encoding the monitor protein; an expression vector for the DNA; a cell expressing the vector; a method of detection/quantitative determination of the monitor protein; and a method of measuring the inhibitory or promotive activity of a test substance against the expression of a membrane protein on the cell membrane or the intracellular transport of a membrane protein.

### BACKGROUND OF THE INVENTION

The present invention relates to a monitor protein capable of measuring the expression of a membrane protein on a cell membrane. The present invention also relates to a DNA encoding the monitor protein; an expression vector for the monitor protein; a cell expressing the monitor protein; a method of detecting/quantitatively determining the monitor protein; and a method of measuring the inhibitory or promotive activity of a test substance against the expression of a membrane protein on a cell membrane and the intracellular transport of a membrane protein.

Cell membrane proteins present on the cell surface are proteins constituting the cell membrane (approximately 4 nanometers in thickness). They are present in various forms, e.g., some proteins are bound to the membrane surface, some proteins are localized on one side of the membrane and embedded, or some proteins are spanning the membrane. Membrane proteins include transporters, ion channels, receptors, enzymes and structural proteins, regulatory proteins and a large number of proteins whose roles are not yet elucidated. There also exist some membrane proteins that are expressed constitutively as cell components of hosts/pathogens.

In multicellular organisms, homeostasis of intracellular and extracellular environments has an essential meaning for their survival. In order to achieve this, organisms exchange substances and information with the external environment via membrane proteins, to thereby maintain the intracellular and extracellular environments constant. For example, ion transporters have a function of performing active transport of inorganic ions (which, in principle, do not permeate lipid bilayer membranes) and a large number of water-soluble organic substances involved in biometabolism, by using energy from ATP.

In order for membrane proteins to function physiologically, they must be localized at specific sites on the cell membrane while accumulating various activity regulatory mechanisms. The integration of membrane proteins at specific sites, accumulation of membrane proteins, and the maintenance of localization are observed in almost all of the researches on membrane transportation. For the expression of the function of a membrane protein, intracellular transport to the cell surface after biosynthesis is indispensable and changes in the amount of expression on the cell surface affects greatly on cellular signal transduction. Therefore, it is important to profile those molecules that change the intracellular transport of membrane proteins.

In order for a membrane protein to exert its function, the protein must be transported to the cell surface after biosynthesis, and the efficiency of this transportation correlates with the amount of expression on the cell surface (Non-Patent Document 1). The process of intracellular transportation of a membrane protein is measured as a transition time from an endoplasmic reticulum to a Golgi body by combining the molecular increase by sugar chain addition and a pulse chase experiment (Non-Patent Document 2).

In the functional analysis of ion channels/transporters, the functional properties of transported proteins are analyzed by examining channel current by the voltage clamp method or patch clamp method (Non-Patent Document 3) and examining transporter activity by transport experiments with membrane vesicles. Recently, necessity for monitoring the normal expression of ion channels/transporters has been increased in the field of drug development. As a result, an automated apparatus for the patch clump method and a monitoring method using atomic absorption spectrometry (Non-Patent Document 4), a method of measuring ion channels using a fluorescent pigment (Non-Patent Document 5), and so on have been developed. However, though accurate data is obtainable by the patch clamp method, the number of samples that can be handled per day by this method is about 3,000 at the best even after automation. While atomic absorption spectrometry is easily applicable to high-through-put analysis, this introduces into cells rubidium ions that are not inherently present therein and may disturb the intracellular proteome. In the method using a fluorescent pigment, it is necessary to apply to test samples UV radiation or laser beam of a specific wave length in order to excite the fluorescent pigment. Thus, a similar problem will occur.

Under circumstances, it has been desired to solve the above-described problems and develop an assay system for measuring extracellular transport of membrane proteins that enables rapid and large-scale screening. In drug development, both are important to discover a substance that improves intracellular transport as a major drug action for treatment and to examine whether or not a substance inhibits intracellular transport as a side effect. However, as stated above, there is no high-through-put system for membrane protein trafficking and, thus, efficient profiling is impossible.

In the process of rapid progress in the identification of genes of ion channel molecules and transporter molecules per se, analysis of physiological functions thereof, analysis of their relation with pathology, etc., necessity for profiling the inhibition/promotion of intracellular transport of membrane proteins and for measuring the normal delivery of membrane proteins to the cell membrane and their functions therein has been increased in the field of drug development. For example, it has been found out that a large number of antiarrhythmic drugs, antihistamines, psychotropic drugs and antibiotics inhibit the expression of hERG (human ether-a-go-go related gene product) on the membrane surface and initiate long QT syndrome. It has been recommended to confirm the safety of every candidate drug on hERG channel.
[Non-Patent Document 1] Pfeffer, S. Cell (2003) 112, 507-517.
[Non-Patent Document 2] Nishimura, N. and Balch, W. E. Science (1997) 277, 556-558.
[Non-Patent Document 3] Hamill, O. P., Marty, A., Neher, E., Sakmann, B. and Sigworth, F. J. Pflugers Arch. (1981) 391, 85-100.
[Non-Patent Document 4] Weir, S. W. and Weston, A. H. (1986) Br. J. Pharmacol. 88, 121-128.
[Non-Patent Document 5] Waggoner; A. J Membr Biol. (1976) 27, 317-334.

### SUMMARY OF THE INVENTION

It is an object of the present invention to prepare a protein capable of measuring the expression of a membrane protein on the cell membrane.

The present inventors have expressed a membrane protein fused to a luminescent protein on the cell surface, reacted a substrate (which does not permeate cells) for the luminescent protein with the fused luminescent protein and measured the amount of the resultant luminescence, to thereby succeed in measuring the expression of the membrane protein on the cell surface. Further, by combining therewith a method of deactivating a protein without damaging the cell function, the present inventors have also succeeded in monitoring the intracellular transport of a membrane protein. The present inventions have been achieved based on these findings.

The subject matters of the present inventions are as described below.
(1) A monitor protein capable of measuring the expression of a membrane protein on a cell membrane, said monitor protein comprising a fusion protein in which the membrane protein and a luminescent protein are linked.
(2) The monitor protein of (1) above, wherein the membrane protein includes an ion channel, a transporter, a G protein, an ionotropic receptor, a receptor tyrosine kinase, a G-protein-coupled receptor or a cell adhesion molecule.
(3) The monitor protein of (1) above, wherein the luminescent protein is a non-secretion type luminescent protein.
(4) The monitor protein of (1) above, wherein the luminescent protein is derived from any organism selected from the group consisting of luminous insects, luminous dinoflagellates (Dinoflagellida), Noctiluca, Renilla, Gauissia, Cavernularia, Cypridina and Aequorea.
(5) The monitor protein of (1) above, which is designed so that the luminescent protein is located in the extracellularly expressed region of the membrane protein.
(6) The monitor protein of (1) above, which is designed so that the luminescent protein is not located in the transmembrane domain of the membrane protein.
(7) The monitor protein of (1) above, which is designed so that the luminescent protein is located on the amino terminal side or the carboxyl terminal side.
(8) The monitor protein of (1) above, comprising a spacer sequence between the membrane protein and the luminescent protein.
(9) A DNA encoding the monitor protein of any one of (1) to (8) above.
(10) An expression vector comprising the DNA of (9) above.
(11) A transformed cell carrying the expression vector of (10) above.
(12) A method of detecting or quantitatively determining the monitor protein of (1) above, comprising contacting the monitor protein with a light-emitting substance and measuring the amount of the resultant luminescence.
(13) The method of (12) above, wherein the light-emitting substance is selected from the group consisting of firefly luciferin, bacterial luciferin, dinoflagellate luciferin, vargulin and coelenterazine.
(14) The method of (12) above, wherein the light-emitting substance has a nature that it does not permeate the lipid bilayer membrane of cells.
(15) A method of measuring the expression of a membrane protein on a cell membrane, comprising using the method of (12) above.
(16) A method of measuring the intracellular transport of a membrane protein, comprising using the method of (12) above.
(17) A method of measuring the inhibitory or promotive activity of a test substance against the intracellular transport of a membrane protein, comprising:
(a) a step of preparing a cell which expresses the monitor protein of (1) above on its cell membrane;
(b) a step of deactivating the monitor protein expressed on the cell membrane of the said cell;
(c) a step of contacting the test substance with said cell; and
(d) a step of contacting a light-emitting substance with said cell and then measuring the time course of the amount of the resultant luminescence.
(18) A method of measuring the inhibitory or promotive activity of a test substance against the expression of a membrane protein on the cell membrane, comprising:
(a) a step of preparing a cell which expresses the monitor protein of (1) above on its cell membrane;
(b) a step of deactivating the monitor protein expressed on the cell membrane of the said cell;
(c) a step of contacting the test substance with said cell; and
(e) a step of contacting a light-emitting substance with said cell and then measuring the amount of the resultant luminescence.

According to the present invention, it becomes possible to measure simply and rapidly the expression of membrane proteins on a cell membrane, to which application of a high-through-put method has been so far impossible.

Further, by incorporating a step of inactivating the fusion protein expressed on the cell membrane, the present inventors have successfully constructed a system for simply and rapidly determining the intracellular transport process of a membrane protein and the time required for reaching the cell membrane.

### BRIEF DESCRIPTION OF THE DRAWINGS

FIG. 1 is a schematic drawing showing forms of existence of the monitor protein on surface of a cell membrane.

FIG. 2 shows structural formulas of luciferins.

FIG. 3 is a schematic drawing of an apparatus which supports the automation of luminescence measurement.

FIG. 4 is a schematic drawing of the structures of DNAs encoding monitor proteins.

FIG. 5 is a graph comparing the luminescence activities of monitor proteins expressed on cell membranes with those of monitor proteins remaining within cells.

FIG. 6 is a graph comparing the luminescence activity of a monitor protein which was expressed on a cell membrane through intracellular transport with that of a monitor protein whose expression on a cell membrane was inhibited by treatment with a reagent that inhibits its intracellular transport.

### LEGENDS

1. Sample
2. Well plate
3. Sample table
4. Motor
5. Suction pump
6. Waste pipeline
7. Waste tank
8. Liquid feeding pump 1
9. Washing solution
10. Liquid feeding pipeline
11. Dispenser
12. Light-emitting substrate
13. Liquid feeding pump 2
14. Condenser
15. Detector
16. Computer

### DETAILED DESCRIPTION OF THE INVENTION AND PREFFERED EMBODIMENTS

Hereinbelow, the present invention will be described in detail.

Membrane proteins occupy 30% of the total protein of organisms and have a biologically important meaning. Specifically, membrane proteins which are involved in the transfer of substances between outside and inside of cells and transfer of extracellular information into cells are indispensable for the maintenance of survival of cells. In order for the function of a membrane protein to be expressed, the protein after biosynthesis must be transported up to the cell surface. It is reported that the efficiency of this transport directly correlates with the amount of expression of the protein on the cell surface. Since changes in the amount of expression on the cell surface greatly affect the signal transduction of cells, it is important to profile those molecules which affect the intracellular transport of membrane proteins. In drug development, both are important to discover a substance that improves intracellular transport as a major drug action for treatment and to examine whether or not a substance inhibits intracellular transport as a side effect. However, as stated above, there is no high-through-put assay system for examining the transport of membrane proteins and, thus, effective profiling is impossible.

The present inventors have created a monitor protein capable of measuring the expression of a membrane protein on a cell membrane simply and, with this monitor protein, have enabled high-through-put analysis of drugs or dug candidate compounds in the field of drug development on their promotive or inhibitory activity against the expression of membrane proteins.

The present invention provides a monitor protein capable of measuring the expression of a membrane protein on a cell membrane, the monitor protein comprising a fusion protein in which the membrane protein and a luminescent protein are linked.

The membrane proteins in the present invention include those proteins which have the substance transport functions of transporters and ion channels present on the cell surface; those proteins which have the information transduction functions of receptors and regulatory proteins; structural proteins which are necessary for maintaining the shape of the cell; and those proteins whose functions are unknown. For example, the membrane proteins in the present invention include, but are not limited to, ion channels, transporters, G proteins, ionotropic receptors, receptor tyrosine kinases, G-protein-coupled receptors and cell adhesion molecules.

The type of the membrane protein which may be analyzed in the present invention is not particularly limited as long as the protein is expressed on a cell membrane. For the preparation of a monitor protein, a preferable membrane protein has one or more transmembrane domains and a part of the protein is exposed outside of the cell.

Membrane proteins have a rich variety in types, and functions of a large number of them are still unknown. However, owing to the genome project, it is possible to predict transmembrane domains and amino acids present outside of the cell even in those membrane proteins for which detailed analysis has not progressed. More preferably, it is contemplated to use those membrane proteins for which physiological function analysis and analysis of relation with pathology have more progressed.

Examples of the luminescent protein include, but are not limited to, luminescent proteins derived from various luminous organisms such as luminous insects (firefly, headlight beetle, etc.), Renilla, dinoflagellates (Dinoflagellida), Noctiluca, Latia neritoides, Gaussia, Cavernularia, Cypridina and Aequorea (e.g., luciferase, green fluorescent protein (GFP)).

The luminescent protein is preferably a non-secretion type protein, because when the luminescent protein has been secreted out of the surface of the cell membrane, it becomes impossible to accurately measure the luminescence activity derived from the monitor protein expressed only on a cell membrane. However, even a secretion type luminescent protein may also be used once it has been linked to a membrane protein to form a monitor protein and began to exist on a cell membrane. Alternatively, a secretion type luminescent protein may be converted into a non-secretion type by deleting its secretion signal sequence.

Further, luminescent proteins whose amino acid sequences have been modified for giving thermal stability, pH resistance, surfactant resistance, etc. may also be used in the monitor protein. The above-described membrane proteins and the luminescent proteins may not necessarily be full-length proteins as long as they retain the nature of membrane proteins or the nature of luminescent proteins.

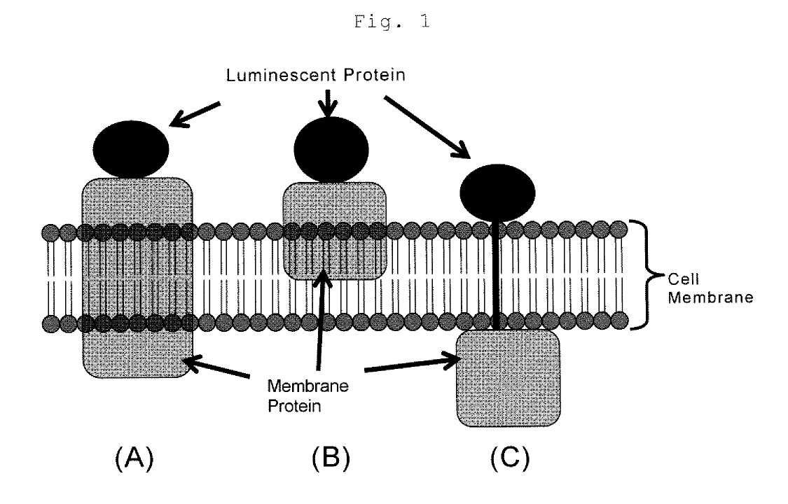

The monitor protein is designed by linking a membrane protein to a luminescent protein so that they function as a fusion protein. In this case, the membrane protein preferably has one or more transmembrane domains. The forms of existence of membrane proteins on a cell membrane are as follows: membrane-spanning proteins; proteins embedded on one side of the membrane; and proteins bound to the surface of the membrane. The monitor protein is designed so that a luminescent protein is fused to a specific site of a membrane protein. It is possible to judge which domain of the membrane protein is exposed to the outside of the cell from literature or based on the results of structural simulation with computer. The monitor protein may be designed so that the luminescent protein is located near the exposed domain of the membrane protein. Even a membrane protein without any domain exposed to the outside of the cell may be made functional as a monitor protein by attaching a luminescent protein to its amino or carboxyl terminal so that the luminescent protein is exposed to the outside. Preferably, the monitor protein is designed so that the luminescent protein is not located in the transmembrane domain of the membrane protein. FIG. 1 shows the forms of existence of the monitor protein on the cell membrane.

Further, in the present invention, it is possible to add known His-tag sequence or FLAG sequence to the amino or carboxyl terminal of the monitor protein so that the expression of the monitor protein can be analyzed by such methods as Western blotting or ELISA. As a negative control for the monitor protein of the invention, a monitor protein may be used to which known organelle transport signals are added at its amino or carboxyl terminal. The most preferable embodiment of the negative control is a monitor protein to which an organelle transport signal (KDEL sequence) is added at its carboxyl terminal.

A leader sequence may be inserted into the monitor protein at its amino terminal. The leader sequence is a sequence involved in the adsorption of a newly born peptide onto a cell membrane and enables the expressed fusion protein to be expressed on the surface of the cell membrane through a membrane transport system.

In the monitor protein of the present invention, the spatial arrangement of the two components (i.e., a membrane protein and a lumninescent protein) may largely affect the performance of the monitor protein. When the membrane protein is fused to the luminescent protein, the following possibilities may be considered depending on the mode of folding of the proteins: the active center of the luminescent protein may not appear outside of the cell; even if the monitor protein is expressed on the cell membrane, the function as a membrane protein may be remarkably damaged; or a stress occurs within the cell and a misfolding occurs in the monitor protein, as a result, the protein is recognized as a foreign substance and may undergo non-specific proteolysis by endocytosis or autophagy.

In order to solve these problems, it is contemplated to insert a peptide sequence (functioning as a spacer) at the binding site of the membrane protein to the luminescent protein. The peptide may consist of 1-100 amino acids. The types of amino acid residues may be selected from those residues which do not easily take a secondary structure, i.e., the residues may be selected from aspartic acid, asparagine, proline, serine and glycine; and yet the peptide may be composed of a single type of amino acid residue. For example, the following sequences may be enumerated.

(SEQ ID NO: 9)

Pro-Pro-Pro-Pro-Pro-Pro-Pro-Pro-Pro-Pro-Pro-Pro-
Pro

(SEQ ID NO: 10)

Gly-Gly-Gly-Gly-Gly

(SEQ ID NO: 11)

Asn-Asn-Asn-Asn-Asn-Asn-Asn-Asn-Asn-Asn
Asp-Asp

(SEQ ID NO: 12)

Ser-Ser-Ser-Ser-Ser-Ser-Ser-Ser-Ser-Ser-Ser-Ser-
Ser-Ser-Ser-Ser-Ser

The present invention also provides a DNA encoding the above-described monitor protein. The DNA may be prepared by obtaining genetic information on the component proteins from gene sequence information databanks such as GenBank, EMBL, DDBJ, etc. and using the known PCR method or a method using restriction enzymes and ligases.

For expressing the monitor protein in cells, it is preferable that the DNA encoding the monitor protein be located under the control of a promoter expressed constitutively in the cell. Examples of constitutively expressed promoters include HSVtk promoter, SV40 promoter, CMV promoter and promoters derived from housekeeping genes such as EF-1α. Further, the present invention provides an expression vector comprising a DNA encoding the above-described monitor protein. This expression vector may be obtained by inserting a DNA encoding the monitor protein of the present invention into a known eukaryote expression vector (e.g., p3×FLAG-CM10 or p3×FLAG-CMV9 from Sigma), prokaryote expression vector (e.g., pET-19b from Merck) or a virus vector (e.g., pLP-Adeno-X-CMV from Clontech).

The present invention further provides a transformed cell and a transgenic animal, both retaining the above-described expression vector. The transformed cell may be obtained by introducing the above-described expression vector into a target cell. As a method of vector introduction into cells, known methods such as transfection or viral infection may be used. Such methods as lipofection or electroporation may be enumerated. As a cell to which the gene is introduced, eukaryotes (e.g., Chinese hamster ovary cell-derived CHO-K1 cell, human embryonic kidney-derived HEK293 cell) or yeast may be enumerated. Alternatively, a prokaryote such as Escherichia coli may be used. Further, transgenic animals may be prepared by a known method of microscopic injection of DNA into the anterior nuclei of fertilized eggs.

The present invention further provides a method of detecting or quantitatively determining the monitor protein, comprising contacting the monitor protein with a light-emitting substance and measuring the amount of the resultant luminescence. By using this method, it is possible to measure the expression of a membrane protein on a cell membrane or to measure the intracellular transport of a membrane protein. For retaining and expressing its function, a protein undergoes transcription/translation and moves through intracellular transport to a place where it should function. The present invention provides a technique for performing the conventionally practiced monitoring of the expression of membrane proteins on a cell membrane in a simple manner.

In order to measure the expression on a cell membrane accurately, the following conditions must be satisfied: (1) the assay measures the activity of only those proteins that are present on the cell surface; (2) the time required for the assay is sufficiently shorter than the time required for functional recovery of the cell; and (3) the functional proteins on the cell surface are quickly, quantitatively and irreversibly inactivated. When these conditions have been satisfied, it becomes possible for the first time to evaluate the function of only those membrane proteins which newly reached the cell surface. The present invention has made it possible to construct a system that satisfies all of the above conditions (1) to (3) and yet is capable of measuring the luminescence activity of those monitor proteins that newly reached the cell surface.

Hereinbelow, one embodiment of the procedures for a method of measuring the expression of membrane proteins on a cell membrane and the intracellular transport rate of membrane proteins will be described.

First, the vector of the present invention is introduced into a target cell by a known method such as transfection or microinjection. The cell is cultured for an appropriate period of time, preferably at least for 24 hours. Subsequently, whether or not the cell into which the expression vector for a monitor protein has been introduced is expressing the monitor protein of the present invention on the cell membrane is measured. Briefly, a luciferin (which does not permeate the cell membrane) is directly added to the medium in which the cell is cultured, followed by measurement of the amount of luminescence.

After confirming that the vector-introduced cell is expressing the monitor protein on the cell membrane by the above-described method, the function of the monitor protein on the cell membrane is eliminated. As a reagent for temporarily eliminating the function of the monitor protein, a reagent which deactivates proteins irreversibly is used. Examples of such a reagent include reagents which chemically modify a specific amino acid residue and antibodies to specific membrane proteins. Alternatively, a proteolytic enzyme (protease) may be used for this purpose. In the present invention, Sulfo-NHS which chemically modifies lysin residues specifically or [2-(trimethylammonium) ethyl] methanethosulfonate (MTSET) which chemically modifies cysteine residues specifically may be used preferably. Further, preferably, the modifying reagent is water-soluble, does not affect other proteins and affects other cell functions as little as possible.

Subsequently, a drug (test substance) to be examined as to whether it may promote or inhibit intracellular transport, or whether or not it may affect the normal expression of membrane proteins is contacted with the cell. The treatment time varies depending on the type of the drug, and may be appropriately selected within a range from 1 minute to 24 hours. The drug may be used alone or it may be mixed with a pharmacologically acceptable carrier. Such drugs may be used independently or in combination. When an animal is used instead of a cell, the drug may be administered orally or parenterally.

In order to measure the luminescence activity (“the amount of luminescence”) using bioluminescence, a light-emitting substance (e.g., luciferin) is added to the drug-treated cell on the cell membrane where the monitor protein is expressed. The term “light-emitting substance” used herein means a concept that includes substances which emit light upon interaction with a luminescent protein. For example, luciferin (a light-emitting substance) generates light upon reaction with luciferase (a luminescent protein). This bioluminescence is caused by oxidation of an organic molecule luciferin with oxygen or one of metabolites. This reaction is catalyzed by a luminescent protein or a protein commonly known as luciferase as a result of tight binding or binding by covalent bond of luciferin to the protein. The scheme of this reaction is as follows.

Oxygen (oxyanion or hydrogen peroxide)+luciferin+luminescent protein (or luminescent enzyme)→oxyluciferin+light

Further, the following factors are necessary for the progress of his reaction.
(a) cations such as H⁺, Ca²⁺ or Mg²⁺; transition metals such as Cu⁺/Cu²⁺ or Fe²⁺/Fe³⁺
(b) cofactors such as ATP, NADH or FMN

To date, five types of luciferins have been identified.
(1) Firefly luciferin
(2) Bacterial luciferin
(3) Dinoflagellate luciferin
(4) Vargulin
(5) Coelenterazine

Firefly luciferin is the luciferin found in fireflies (North American firefly, headlight beetle, Phrixothrix hirtus, Rhagophthalmidae ohbai, etc.) and is the substrate for luciferase (EC 1.13.12.7). It is a benzothiazol derivative.

Bacterial luciferin is a type of luciferin found in bacteria, some squid and fish. It has a structure consisting of a long-chain aldehyde and a reduced riboflavin phosphate.

Dinoflagellate luciferin is a chlorophyll derivative and has a tetrapyrrole ring. This luciferin is mainly isolated from dinoflagellates (marine planktons). A similar type of luciferin is found in some types of euphausiid shrimp.

Vargulin is found in ostracods and Midshipman fish. It is an imidazolopyrazine derivative.

Coelenterazine is found in radiolarians, ctenophores, cnidarians, squid, copepods, chaetognaths, fish and shrimp. It is the light-emitting molecule in the luminescent protein aequorin.

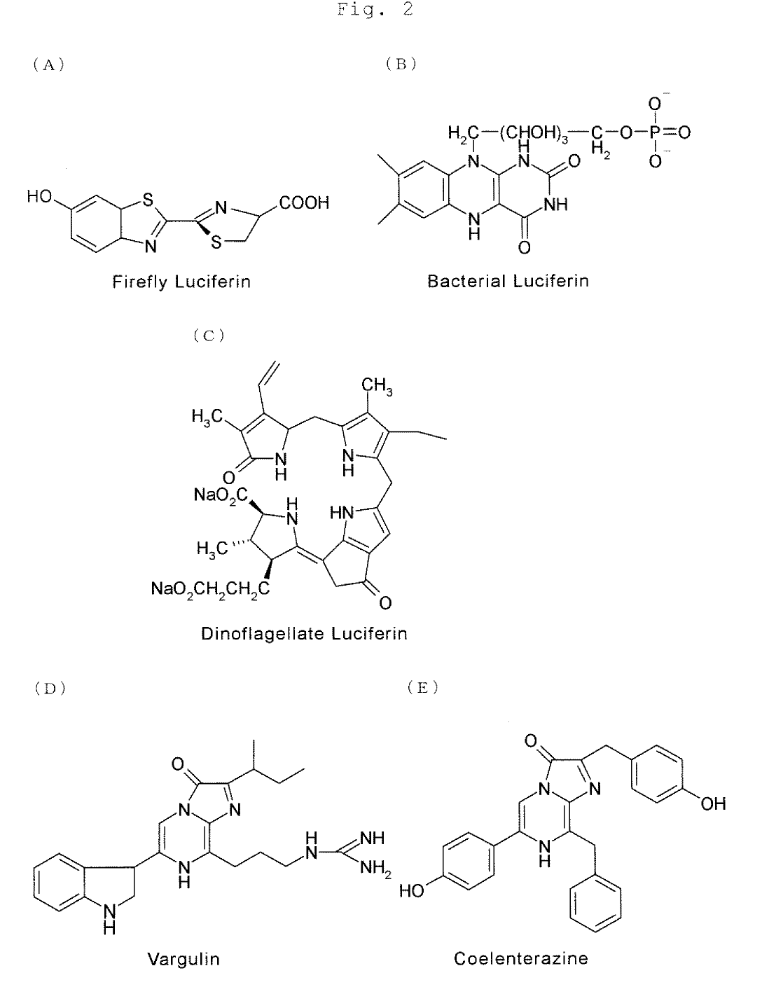

The chemical structures of the representative luciferins in the above five types are summarized in FIG. 2. For further description on light-emitting substrates, see J. W. Hastings, “biological diversity, chemical mechanisms, and the evolutionary origins of bioluminescent systems” (1983, Journal of Molecular Evolution. v. 19: pp. 309-321).

For measuring the expression of the monitor protein of the invention on the cell membrane using luminescent reactions, every types of luciferins mentioned above may be used. The luciferin frequently used in reporter assays at present is firefly luciferin or coelenterazine. It is known that these luciferins are absorbed into cultured cells. Since the assay of the present invention is characterized by measuring only the activity of proteins present on the cell surface, it is preferable to be able to measure only the luminescence activity of the monitor protein expressed on the cell membrane. Therefore, when firefly luciferase or Renilla luciferase, for example, is used as a component of the monitor protein, their substrates may be absorbed into the cell and may allow the monitor protein in the middle of intracellular transport to emit light.

In order to avoid this problem, a light-emitting substance that does not permeate the lipid bilayer of the cell may be used so that the substance is not absorbed into the cell. As a light-emitting substance with such a nature, dinoflagellate (Dinoflagellida) luciferin may be given (panel C, FIG. 2).

Therefore, as a luminescent protein (one component of the monitor protein of the present invention), it is preferable to use a luciferase derived from dinoflagellate (Dinoflagellida) whose substrate is a straight-chain tetrapyrrol luciferin which has been found not to permeate the cell membrane.

A light-emitting substance such as luciferin may be added alone when the monitor protein is measured. Alternatively, drugs which protect luciferin may be added simultaneously with luciferin. Preferable examples of drugs which protect luciferin include sulfhydryl compounds such as dithiothreitol, dithioerythritol, β mercaptoethanol, 2-mercaptopropanol, 3-mercaptopropanol, 2,3-dithiopropanol, glutathione, coenzyme A; and vitamins such as ascorbic acid and α-tocopherol.

When the luminescence enzyme needs cofactors, they may be added simultaneously with the light-emitting substance.

The light-emitting substance may be added to the medium. Alternatively, it is contemplated that the addition thereof is supported by an automated system.

In order to introduce a light-emitting substance into a transgenic animal or plant, a method may be used in which the light-emitting substance is spread throughout the organism by perfusion.

In order to measure the luminescence activity, preferably, the number of photons is measured with a commonly used photomultipler and then converted into numerical values with an instrument called luminometer. When a cultured cell is used, these measuring procedures may be automated.

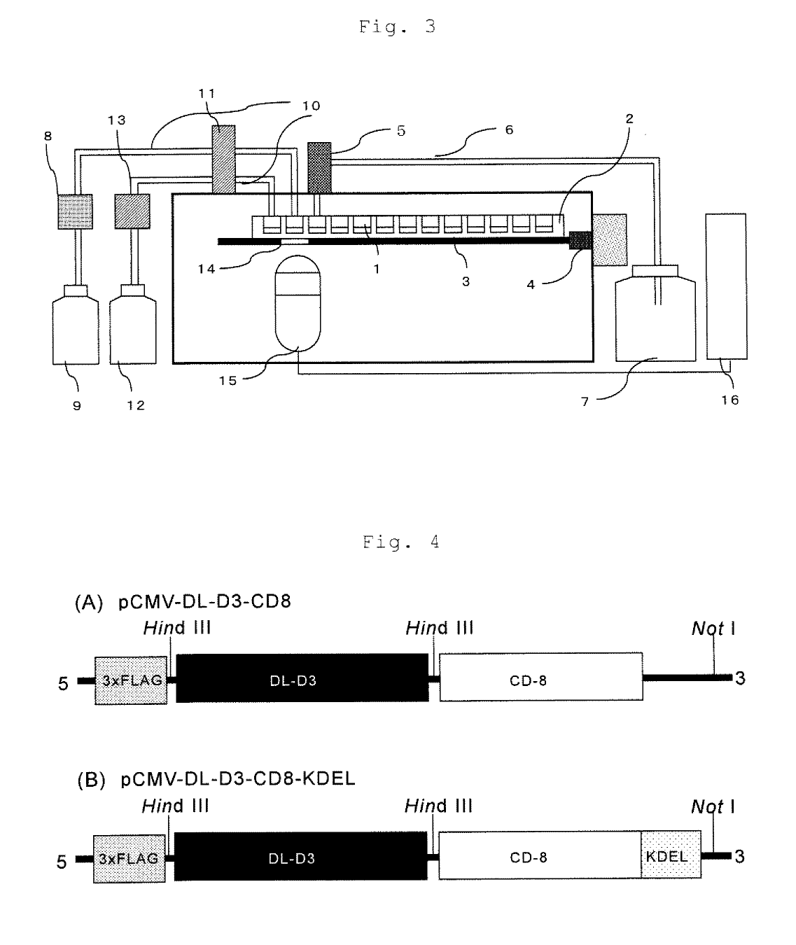

FIG. 3 shows a schematic drawing of one example of an apparatus that supports the automation of measurement of luminescent activity when a cultured cell is used. A well-plate (2) in which a cell strain (sample (1)) that was contacted with a test substance and left for a specific period of time is cultured is placed on a sample table (3). The position of wells is set by horizontally moving the sample table with a motor (4). Since the medium contains substances which affect luminescent reactions (drugs that deactivate membrane proteins and test substances), it is desirable to remove the medium. The medium is removed by a suction pump (5) and fed to a waste pipeline (6). The waste pipeline (6) is connected to a waste tank (7). Subsequently, in order to completely remove the medium and test substance adhering to the well wall, wells are washed. A washing solution (9) is fed by liquid feeding pump 1 (8) to a dispenser (11) through a liquid feeding pipeline (10), and divided into portions, which are then fed to wells. The amount of the portion may be regulated by computer and changed at discretion depending on the size of the well. As the washing solution, physiological saline or PBS buffer may be used. In order to remove the washing solution dispensed into wells, the washing solution (9) is fed to the waste pipeline (6) by the suction pump (5). In order to measure the amount of luminescence of the luminescent protein expressed on the cell membrane, a light-emitting substrate (12) is dispensed into wells. The light-emitting substrate (12) is fed by liquid feeding pump 2 (13) to the dispenser (11) through the liquid feeding pipeline (10). The dispenser (11) is capable of controlling at discretion the amount to be dispensed. It is contemplated that the light-emitting substrate is selected depending on the type of luminescent protein so that it takes the form of a detection reagent with optimized reaction conditions. The light emitted from the sample is condensed by a condenser (14) and introduced into a detector (15). Luminescent signals are converted into electric signals in the detector and sent to a computer (16). It should be note here that the photomultipler, sample table, sample and condenser are contained in a box made of a material capable of completely blocking the light from the outside.

It is also contemplated that luminescence on the cell surface is confirmed by exposing to X-ray films and quantitatively determining the luminous intensity from the images or by using a CCD camera or a microscope capable of detecting luminescence. When membrane proteins in animal or plant bodies are measured, use of a CCD camera is particularly preferred.

When the inhibitory or promotive activity of a drug (test substance) against the expression of a membrane protein on the cell membrane is measured, the above-described cell treated with the drug (the monitor protein expressed on the cell membrane has already been deactivated) may be contacted with a light-emitting substance, and then measurement may be performed after an appropriate time has passed. For example, as a technique to confirm the presence or absence of the inhibitory or promotive effect of a drug against the expression of a membrane protein on the cell membrane, the amounts of luminescence in the drug-treated plot and untreated plot may be measured and compared. Briefly, when the amount of luminescence in the drug-treated plot is less than the amount of luminescence in untreated plot, it can be said that the drug inhibits the expression of the membrane protein. On the contrary, when the amount of luminescence in the drug-treated plot is more than the amount of luminescence in untreated plot, it can be said that the drug promotes the expression of the membrane protein. The measurement of the amount of luminescence may be performed after a sufficient time has passed from the deactivation treatment of the monitor protein, so that new monitor protein is allowed to be expressed on the cell membrane.

Further, in another embodiment, the above-described cell may be solubilized to thereby liberate the cell contents in the medium. Then, a light-emitting substance such as luciferin may be added to the medium, and the amount of luminescence may be measured. Examples of components for solubilization include Triton-X100 and saponin. Preferable concentrations of Triton-X100 and saponin are 0.1-1% and 0.01-0.1%, respectively (concentrations at the time of assay). By these procedures, it is possible to measure the amounts of luminescence of the monitor protein expressed inside of the cell and the monitor protein expressed on the surface of the cell membrane. Using the resultant values, the expression ratio of the monitor protein expressed on the cell membrane may be calculated.

When the inhibitory or promotive activity of a drug (test substance) against the intracellular transport of a membrane protein is measured, the above-described cell treated with the drug (the monitor protein expressed on the cell membrane has already been deactivated) may be contacted with a light-emitting substance, and then measurement may be performed for a specific period of time at appropriate intervals. For example, as a technique to confirm the presence or absence of the inhibition or promotion by a drug against the intracellular transport of a membrane protein, the amounts of luminescence in the drug-treated plot and untreated plot may be measured after passage of any length of time and compared. Briefly, when the amount of change in luminescence (recovery) in the drug-treated plot is less than the amount of change in luminescence in untreated plot, it can be said that the drug inhibits the intracellular transport of the membrane protein. On the contrary, when the amount of change in luminescence (recovery) in the drug-treated plot is more than the amount of change in luminescence in untreated plot, it can be said that the drug promotes the intracellular transport of the membrane protein.

So far, the method of the present invention has been described taking the measurement of the drug action of drugs (test substances) as examples. When the above-described operations are performed without adding such drugs, it is possible to analyze the expression of membrane proteins and intracellular transport thereof. It should be noted that there may be some cases where the treatment to eliminate the function of the monitor protein on the cell membrane is unnecessary in order to analyze the expression of the membrane protein on the cell membrane.

### EXAMPLES

Hereinbelow, the effect of the present invention will be described more specifically with reference to the following Examples. These Examples are not intended to limit the scope of the present invention.

### Example 1

A monitor protein for confirming the expression of a membrane protein on a cell membrane by a luminescent reaction has been designed. As a membrane protein whose expression on the cell membrane is to be confirmed, CD-8 was selected. CD-8 is a molecule of the immunoglobulin superfamily. It is a transmembrane glycoprotein expressed in “suppressor/cytotoxic” T cell subgroup of adult T cells. CD-8 is found in about 63% of thymocytes, about 9% of splenocytes, about 20% of lymph node cells, and about 15% of peripheral blood lymphomas. The function thereof is a costimulatory receptor for MHC (specific)-restricted TCR in the antigen recognition of T cells. As the luminescence protein in the monitor protein, a dinoflagellate-derived luciferase PL-D3 was selected. The monitor protein was designed so that the luminescence protein is located more close to the amino terminal than the membrane protein (D-8). Then, a DNA designed to express the monitor protein was prepared.

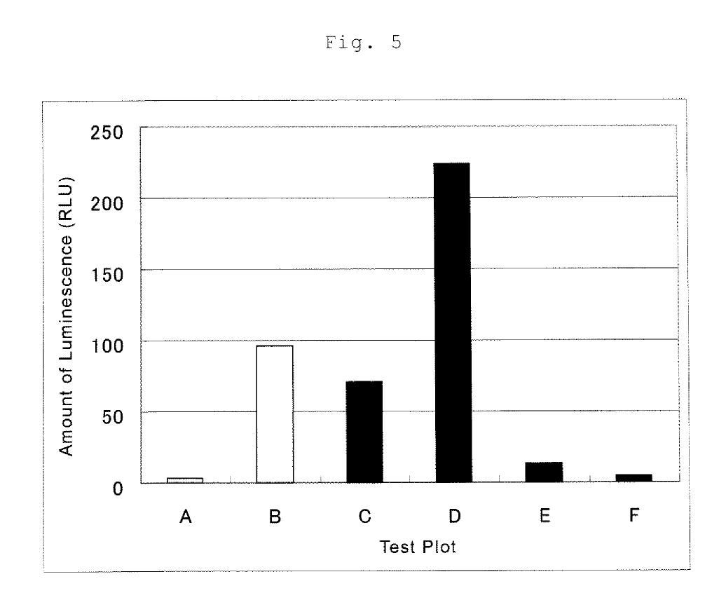

A DNA fragment encoding CD-8 (Gene Bank accession No. NM^{—}001768) (SEQ ID NO: 1) was inserted into the Hind III-Not I site of an animal cell expression vector p3×FLAG-CMV9 (Sigma) by ligation. Ligation was performed using DNA Ligation Kit Ver. 2 (Takara Bio). Since this p3×FLAG-CMV9 vector has a CMV promoter, genes located downstream of this promoter are expressed constitutively in animal cells to which this vector has been introduced. This vector also comprises a leader sequence encoding an amino terminal domain of secretory proteins (an amino acid sequence spanning from position 4 (Ser) to position 17 (Ala) of the amino acid sequence shown in SEQ ID NO: 6). Further, this vector also comprises FALG tag sequence (an amino acid sequence spanning from position 18 (Asp) to position 39 (Lys) of the amino acid sequence shown in SEQ ID NO: 6). Therefore, it is possible to detect the expression of the gene of interest by Western blotting using anti-FLAG antibody. CD-8-inserted p3×FLAG-CMV9 vector is designated pCMV-CD-8. Subsequently, in order to insert dinoflagellate-derived luciferase PL-D3 into the Hind III site of pCMV-CD-8, a DNA fragment of PL-D3 (Gene Bank accession No. AF394059) (SEQ ID NO: 3) was amplified by PCR so that its 5′ and 3′ terminuses have a Hind III site. The amplified PCR fragment was subjected agarose gel electrophoresis. The fragment of interest was cut out and inserted into the Hind III site of pCMV-CD-8. For confirmation of the introduction of PL-D3, a DNA fragment was extracted with MiniPrep and digested with Hind III to thereby confirm the introduction of PL-D3. In order to examine whether mutations are introduced into the sequence of the amplified PL-D3 fragment, sequencing reactions were performed to thereby confirm the nucleotide sequence. The vector in which the correct nucleotide sequence was confirmed was designated pCMV-DL-D3-CD8. The sequence encoding the monitor protein moiety of this vector is shown in SEQ ID NOS. 5 and 6. Further, organelle transport signal (KDEL sequence) (SEQ ID NOS: 7 and 8) was added to the carboxyl terminal side of CD-8, and a monitor protein expression vector was also prepared by the same technique as described above. This vector was designed so that it is not transported and remains in the organelle after transcription/translation in the cell. This vector was designated pCMV-DL-D3-CD8-KDEL. FIG. 4 shows schematic drawings of DNAs encoding the monitor protein in vectors pCMV-DL-D3-CD8 and pCMV-DL-D3-CD8-KDEL.

Subsequently, the two monitor protein expression vectors prepared above were introduced into animal cells. Briefly, CHO-K1 cells (purchased from RIKEN cell bank) were seeded on 6-well plates at 1×10₆ cells/well and cultured in D-MEM/Ham's F-12 medium (Wako Purechamical Industries) containing 10% FCS. After overnight culture at 37° C., 4 μg of pCMV-DL-D3-CD8 or 4 μg of pCMV-DL-D3-CD8-KDEL was added to each well and mixed with 250 μl of Opti-MEM serum-free medium (Gibco RRL) and reacted at room temperature for 5 min. Then, 10 μl of lipofectamine 2000 (Invitrogen) reacted in 250 μl of Opti-MEM serum-free medium at room temperature for 5 min was mixed with the cells to perform transfection in Opti-MEM serum-free medium. Then, the medium was exchanged for D-MEM/Ham's F-12 medium containing 10% FCS. The cells were subcultured to 10 cm dishes, and cultured for another three days. Subsequently, the transfected cells were scraped off with a cell scraper and washed with PBS. Then, cells were counted, followed by preparation of a cell suspension with a cell density of 1×10⁶ cells/ml.

The amounts of luminescence were measured in the monitor protein-expressing cells and cells without the expression vector after adjusting the cell counts. Briefly, to 50 μl of cell suspension, 50 μl of 200 mM phosphate buffer pH 5.5 and 2 μl of 50 μM dinofagellate-derived luciferin (the dinoflagellate-derived luciferin was extracted and purified according to the method described in Japanese Unexamined Patent Publication No. 2005-049213). The number of photons emitted from the sample was measured with Berthold luminometer Centro LB960. Further, in order to examine the amount of luminescence of the monitor protein which is believed remaining in the cell, in addition to the monitor protein expressed on the cell membrane, 50 μl of 200 mM phosphate buffer pH 5.5 containing 0.02% saponin and 2 μl of 50 μM dinoflagellate-derived luciferin were added to 50 μl of cell suspension to allow the cell contents to ooze out. Then, the luminescence activity of this monitor protein was obtained.

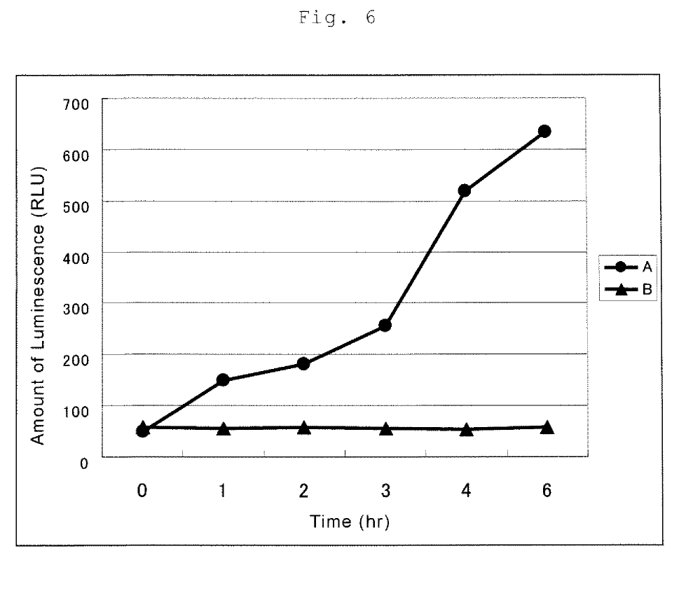

The results are shown in FIG., 5. Luminescence was confirmed in a test plot where pCMV-DL-D3-CD8 was expressed and saponin treatment was not performed (C in FIG. 5). In pCMV-DL-D3-CD8-KDEL which was designed to remain in the organelle, luminescence was background level (A in FIG. 5). Therefore, it was demonstrated that the luminescence confirmed in the above test plot was derived only from the luminescence of the motor protein expressed on the cell membrane. In the saponin-treated plots (B and D in FIG. 5), increase in the amount of luminescence was observed compared to non-treatment plot. These results suggest that the monitor protein in the middle of translation or transport is present in cells and that when the light-emitting substrate has a nature of permeating the cell membrane, this may become a cause to increase the background value. In test plots where neither gene is introduced, luminescence was not confirmed regardless of the presence or absence of saponin treatment (E and F in FIG. 5).

### Example 2

A model experiment using the monitor protein of the present invention was carried out to demonstrate that it is possible to monitor the intracellular trafficking of a membrane protein. The vector pCMV-DL-D3-CD8 prepared in Example 1 was introduced into an animal cell. Briefly, CHO-K1 cells were seeded on 6-well plates at 1=10⁶ cells/well and cultured in D-MEM/Ham's F-12 medium (Wako Purechamical Industries) containing 10% FCS. After overnight culture at 37° C., 4 μg of pCMV-DL-D3-CD8 was added to each well and mixed with 250 μl of Opti-MEM serum-free medium (Gibco RRL) and reacted at room temperature for 5 min. Then, 10 μl of lipofectamine 2000 reacted in 250 μl of Opti-MEM serum-free medium at room temperature for 5 min was mixed with the cells to perform transfection in Opti-MEM serum-free medium. Then, the medium was exchanged for D-MEM/Ham's F-12 medium containing 10% FCS. The cells were subcultured to 10 cm dishes, and cultured for another three days. Subsequently, the transfected cells were scraped off with a cell scraper and washed with PBS. Then, cells were counted, followed by preparation of a cell suspension with a cell density of 2×10⁷ cells/ml.

By adding 0.5 mM Sulfo-NHS (Pierce) to the cell suspension, the monitor protein expressed on the cell membrane was inactivated. Subsequently, an operation to remove the Sulfo-NHS was carried out. Briefly, D-MEM/Ham's F-12 medium containing 10% FCS was added to 10 ml of the monitor protein-inactivated cell suspension, and the resultant mixture was centrifuged at 1,000×g. The precipitate was re-suspended in 22 ml of D-MEM/Ham's F-12 medium containing 10% FCS. Subsequently, a test plot treated with 1 mM Brefeldin A (BFA: BIOMOL) (a reagent that inhibits intracellular transport) and a non-treatment plot were prepared. 1, 2, 3, 4 and 6 hours after the start of culture, cells of BFA-treatment plot and non-treatment plot were suspended in PBS and centrifuged at 1,000 ×g in order to remove dead cells and the dead cell-derived monitor protein contained in the medium. Cells were counted and arranged to give a density of 4×10⁵ cells per 100 μl. This cell suspension was divided into 20 μl aliquots. To one aliquot, 2 μl of dinoflagellate-derived luciferin was added and the luminescence activity of the monitor protein expressed on the cell membrane was measured. Other aliquots were subjected to measurement of the amount of ATP-derived luminescence in order to examine whether the cell count is constant in individual samples taken at the above-indicated time points.

FIG. 6 shows a graph on which the amounts of luminescence derived from the membrane protein expressed on the cell membrane are plotted. In the plot where cells were not treated with BFA, luminescence began to be recognized 1 hour after the deactivation of the membrane protein (line A with filled circles, FIG. 6). However, in the test plot treated with BFA, luminescence was not recognized even 6 hours after the deactivation treatment (line B with filled triangles, FIG. 6). As a result, it was demonstrated that the monitor protein of the present invention enables monitoring of the effect of a drug upon intracellular trafficking by measuring the recovery time after the deactivation treatment of the monitor protein expressed on the cell membrane.

The present invention may be used in the analysis of the expression and the intracellular trafficking of membrane proteins, as well as the measurement of promotion/inhibition of the expression of membrane proteins and promotion/inhibition of intracellular transport thereof. The present invention is also applicable to the screening of drug candidates and prediction of adverse effects.

The entire disclosure of Japanese Patent Application No. 2007-011163 filed on Jan. 22, 2007 including specification, claims, drawings and summary is incorporated herein by reference in its entirety.

All publications, patents and patent applications cited herein are incorporated herein by reference in their entirety.

1. A monitor protein for measuring the expression of a membrane protein on a cell membrane, said monitor protein comprising a fusion protein in which the membrane protein and a luminescent protein are linked.

2. The monitor protein according to claim 1, wherein the membrane protein includes an ion channel, a transporter, a G protein, an ionotropic receptor, a receptor tyrosine kinase, a G-protein-coupled receptor or a cell adhesion molecule.

3. The monitor protein according to claim 1, wherein the luminescent protein is a non-secretion type luminescent protein.

4. The monitor protein according to claim 1, wherein the luminescent protein is derived from any organism selected from the group consisting of luminous insects, luminous dinoflagellates (Dinoflagellida), Noctiluca, Renilla, Gaussia, Cavernularia, Cypridina and Aequorea.

5. The monitor protein according to claim 1, which is designed so that the luminescent protein is located in the extracellularly expressed region of the membrane protein.

6. The monitor protein according to claim 1, which is designed so that the luminescent protein is not located in the transmembrane domain of the membrane protein.

7. The monitor protein according to claim 1, which is designed so that the luminescent protein is located on the amino terminal side or the carboxyl terminal side.

8. The monitor protein according to claim 1, comprising a spacer sequence between the membrane protein and the luminescent protein.

9. An isolated DNA encoding the monitor protein according to any one of claims 1 to 8.

10. An expression vector comprising the isolated DNA according to claim 9.

11. A transformed cell carrying the expression vector according to claim 10.

12. A method of detecting or quantitatively determining the monitor protein according to claim 1, comprising contacting the monitor protein with a light-emitting substance and measuring the amount of the resultant luminescence.

13. The method according to claim 12, wherein the light-emitting substance is selected from the group consisting of firefly luciferin, bacterial luciferin, dinoflagellate luciferin, vargulin and coelenterazine.

14. The method according to claim 12, wherein the light-emitting substance has a nature that it does not permeate the lipid bilayer membrane of cells.

15. A method of measuring the expression of a membrane protein on a cell membrane, comprising using the method according to claim 12.

16. A method of measuring the intracellular transport of a membrane protein, comprising using the method according to claim 12.

17. A method of measuring the inhibitory or promotive activity of a test substance against the intracellular transport of a membrane protein, comprising:
(a) a step of preparing a cell which expresses the monitor protein according to claim 1 on its cell membrane;
(b) a step of deactivating the monitor protein expressed on the cell membrane of the said cell;
(c) a step of contacting the test substance with said cell; and
(d) a step of contacting a light-emitting substance with said cell and then measuring the time course of the amount of the resultant luminescence.

18. A method of measuring the inhibitory or promotive activity of a test substance against the expression of a membrane protein on the cell membrane, comprising:
(a) a step of preparing a cell which expresses the monitor protein according to claim 1 on its cell membrane;
(b) a step of deactivating the monitor protein expressed on the cell membrane of the said cell;
(c) a step of contacting the test substance with said cell; and
(e) a step of contacting a light-emitting substance with said cell and then measuring the amount of the resultant luminescence.
